(19) United States
(12) Patent Application Publication
(10) Pub. No.: US 2013/0197089 A1
(43) Pub. Date: Aug. 1, 2013

(54) COMPOSITIONS FOR THE TREATMENT OF ACTINIC KERATOSIS

(71) Applicant: Juergen Warnecke, Oldenborstel (DE)

(72) Inventor: Juergen Warnecke, Oldenborstel (DE)

(73) Assignee: DOLORGIET GMBH & CO. KG, Sankt Augustin (DE)

(21) Appl. No.: 13/753,783

(22) Filed: Jan. 30, 2013

(30) Foreign Application Priority Data
Jan. 30, 2012 (EP) 12153082.8

(51) Int. Cl. A61K 31/192; A61K 31/196

(52) U.S. Cl. CPC A61K 31/192; A61K 31/196
USPC 514/567; 514/570

(57) ABSTRACT

Ibuprofen can be used in the prevention and treatment of actinic keratosis.

### CLAIM OF PRIORITY

This application claims priority to European application no. EP 12153082.8, filed Jan. 30, 2012, which is incorporated by reference in its entirety.

### TECHNICAL FIELD

The present invention relates to compositions for the treatment of actinic keratosis.

### BACKGROUND

Actinic keratosis is a chronic damage to the keratinized epidermis caused by many years of intensive exposure to sunlight or ultraviolet radiation. Actinic keratosis is a facultative precancerosis and can transform into a squamous cell carcinoma.

Actinic keratosis occurs mainly among people in the second half of life, wherein skin areas that were exposed to sunlight without protection are particularly often affected, for example, the face, the back of the hands, the forehead, the nose or ear. In addition to humans, animals such as dogs and cats may also be affected in sparsely haired areas.

Typical treatment forms include surgical removal or treatment with, for example, imiquimod, 5-fluorouracil, hyaluronic acid with diclofenac, or photodynamic therapy with 5-aminolevulinic acid.

Especially in the region of the face, or when the disease affects larger areas, surgical methods may be undesirable from a cosmetic point of view. Therefore, topical treatment with gels and creams is preferred especially in the face. Currently, treatment with a combination of diclofenac/hyaluronic acid is best tolerated.

### SUMMARY

It is the object of the present invention to provide further possible treatments for actinic keratosis.

This object is achieved by the use of ibuprofen in the prevention and treatment of actinic keratosis.

In one aspect, a method for the prevention and treatment of actinic keratosis includes administering an effective amount of ibuprofen to a patient in need thereof.

Administering can include applying a topical formulation including ibuprofen. The topical formulation can be in the form of an ointment, a cream, or a gel. The concentration of ibuprofen can be from 1 to 20% by weight; from 5 to 10% by weight; or from 6 to 8% by weight.

The topical formulation can further include a penetration enhancer selected from the group consisting of dimethyl isosorbide, poloxamers, laurocapram, pyrrolidones, dimethylsulfoxide, oleic acid, ethylene glycols, derivatives thereof, and mixtures thereof.

In some embodiments, ibuprofen can be the only active ingredient in the topical formulation. In other embodiments, the topical formulation further includes diclofenac.

In some embodiments, the topical formulation does not include any hyaluronic acid.

In another aspect, a pharmaceutical preparation includes a topical formulation including from 6 to 8% by weight of ibuprofen and from 2 to 15% by weight of one or more penetration enhancers.

The one or more penetration enhancers can include dimethyl isosorbide. The topical formulation can be in the form of a gel.

### DETAILED DESCRIPTION

Ibuprofen is an active substance that has been used for many years in the treatment of arthrosis, sports injuries, and rheumatoid diseases. In addition to systemic application, corresponding topical forms of application are also known.

Plasma levels on the order of 10 μg/ml or more are necessary for a general systemic effect to be achieved. In topical application, plasma levels of only about 2 μg/ml are reached. Therefore, topical application does not cause systemic effects, and the side effects are also extremely low.

Therefore, according to the invention, ibuprofen is preferably employed in a topical formulation.

Particularly suitable formulations include ointments, creams and gels. A cream is an emulsion of an aqueous phase in a lipophilic phase; a gel is a disperse system of solid and liquid phases.

Concentrations of ibuprofen in a topical formulation of from 1 to 20% by weight have proven particularly suitable, concentrations of 5 to 10% by weight being particularly preferred. Preferably, the concentration is more than 5% or at least 6% or 7% by weight. The concentration is preferably around 10% by weight or below 10% by weight, for example, up to 9% by weight or up to 8% by weight. The range of from 5 to 10% by weight or from 6 to 8% by weight is preferred, in particular, for gel formulations.

In one embodiment, the formulation additionally contains penetration enhancers, which facilitate penetration through the skin. Such penetration enhancers are known to the skilled person in principle, including from the production of ibuprofen-containing topical formulations for the treatment of sports injuries, for example.

Particularly suitable formulations contain dimethyl isosorbide, poloxamers, laurocapram, pyrrolidones, dimethylsulfoxide, oleic acid, ethylene glycols and derivatives and mixtures thereof as penetration enhancers.

In one embodiment of the invention, ibuprofen is the only active ingredient in the formulation. Of course, ibuprofen requires auxiliary agents for the preparation of an administrable medicament, depending on the dosage form. However, no other active ingredients are contained in this embodiment.

In particular, in one embodiment, no hyaluronic acid is contained, which could be an auxiliary agent or an active ingredient.

In another embodiment, diclofenac is contained in addition to ibuprofen. Preferred amounts of diclofenac are also within a range of from 1 to 20% by weight, concentrations of from 3 to 10% by weight being preferred. Preferably, the total concentration of ibuprofen and diclofenac should be within a range of from 1 to 20%, preferably 5 to 10%, by weight.

A preferred gel formulation (microemulsion gel) contains:
5 to 10 g of ibuprofen;
10 to 25 g of isopropanol;
5 to 10 g of dimethyl isosorbide;
6 to 18 g of poloxamer;
1 to 4 g of medium-chain triglycerides;
perfumes;
water ad 100 g.

Another preferred gel contains:
5 to 10 g of ibuprofen;
30 to 60 g of ethanol 96%;
5 to 20 g of dimethyl isosorbide;
1 to 4 g of hydroxypropylcellulose;
0.75 to 4 g of potassium hydroxide;
water ad 100 g.

The invention is further illustrated by means of the following Examples.

### EXAMPLES

In a preliminary study, the treatment of actinic keratosis using ibuprofen is compared with a treatment using diclofenac. The study was performed according to the relevant criteria and was approved by the ethics commission of the dermatological society of Slovakia.

The study included 24 patients who showed five or more lesions in the area of the head, the face, the arms, or the hands.

12 of the patients were treated with 5% ibuprofen gel, and the others were treated with 3% diclofenac gel. The application frequency was twice a day. The success of the treatment was evaluated after 30, 60 and 90 days.

When included in the study, the severity of the lesions was evaluated according to the so-called BSI (baseline severity index) score from 0 to 3. Patients having a BSI score of 1 and 2 were exclusively included in the study.

BSI score (baseline severity index score 1-3)
0=no actinic keratosis (AK) visible
1=clearly visible lesion
2=many small, moderately thick lesions or a few large scaly lesions visible
3=many thick hypertrophic lesions that are clearly visible and palpable with clear bounds.

The main target parameters within the scope of effectiveness were the total number of lesions in the treatment area and the IGII (investigator global improvement index) score from −2 to +4. From round 1, the evaluations were always done in comparison with the preliminary round.

TABLE 1
Average number of lesions at the start of the study

| | Ibuprofen | Diclofenac |
|---|---|---|
| Forehead | 4.6 | 7.1 |
| Face | 5.7 | 7 |
| Scalp | 6.3 | 15.8 |
| Hands | 18.5 | 18.1 |

TABLE 2
Average number of lesions after 90 days of treatment

| | Ibuprofen | Diclofenac |
|---|---|---|
| Forehead | 4 | 5.3 |
| Face | 4.8 | 5.5 |
| Scalp | 6 | 11.5 |
| Hands | 16.9 | 17.1 |

After 90 days of treatment, the average number of lesions was reduced from 35.1 to 31.7 in the ibuprofen group and from 48 to 39.4 in the diclofenac group. The severity of the lesions remained virtually unchanged during the 90 days of treatment. A small reduction of the BSI score occurred only on the forehead. None of the patients developed new lesions during their treatment.

IGII (investigator global improvement index)
−2 significant worsening
−1 slight worsening
0 unchanged
1 slight improvement
2 improvement
3 significant improvement
4 complete healing

The IGII was measured between each two rounds. It may take a value of −2 (significant worsening) to +4 (healing) and changed as follows during the 90 days of treatment:

TABLE 3
Investigator global improvement index at the start of
the study (V0) and after 90 days of treatment (V3)

| | |
|---|---|
| Ibuprofen | Diclofenac |

| | V0 | V3 | V0 | V3 |
|---|---|---|---|---|
| Forehead | 0 | 1.33 | 0 | 1.55 |
| Face | 0 | 1.38 | 0 | 1.7 |
| Scalp | 0 | 1 | 0 | 1.75 |
| Hand | 0 | 1 | 0 | 1 |

### Result

In both groups, a reduction in the number of lesions could be seen, which, although it was only slight, in combination with the positive change of the IGII score underlines an about comparable effectiveness of the two treatments (ibuprofen and diclofenac). The differences in the reduction of the lesions when comparing ibuprofen and diclofenac are essentially due to the number of lesions at the start of the study. No undesirable side effects were found during the study. The therapy was well tolerated.

In another study, six patients were treated with 10% ibuprofen gel, and another six patients were treated with 3% diclofenac gel. The design of the study was the same as that of the first study.

The total number of lesions in the ibuprofen group changed from 18 at the beginning of the treatment to 3.7 at the end of the 90 days of treatment time. In the diclofenac group, there were 36.8 lesions at the start, which changed to an average of 19.2 lesions towards the end of the treatment.

TABLE 4
Average number of lesions at the start of the study

| | Ibuprofen | Diclofenac |
|---|---|---|
| Forehead | 3.3 | 7.5 |
| Face | 5.2 | 12.3 |

| | |
|---|---|
| Scalp | No evaluation since only 1 patient showed lesions |

| | | |
|---|---|---|
| Hands | 9.5 | 17 |

TABLE 5
Average number of lesions after 90 days of treatment

| | Ibuprofen | Diclofenac |
|---|---|---|
| Forehead | 0.3 | 4.2 |
| Face | 0.7 | 6.2 |

| | |
|---|---|
| Scalp | No evaluation since only 1 patient showed lesions |

| | | |
|---|---|---|
| Hands | 2.7 | 8.8 |

The average IGII at the end of the treatment time was 3.7 (i.e., the lesions were almost completely healed) in the ibuprofen group and 2.29 in the diclofenac group, which corresponds to a moderately successful healing.

TABLE 6
Investigator global improvement index at the start of
the study (V0) and after 90 days of treatment (V3)

| | Ibuprofen | | Diclofenac | |
|---|---|---|---|---|
| | V0 | V3 | V0 | V3 |
| Forehead | 0 | 3.8 | 0 | 2.3 |
| Face | 0 | 3.7 | 0 | 2.17 |
| Scalp | No evaluation since only 1 patient showed lesions | | | |
| Hand | 0 | 3.6 | 0 | 2.4 |

### Result

Even if the small number of patients in the 2nd study is considered, the successful treatment in the 10% ibuprofen group shows nevertheless that the formulation has a pronounced effectiveness in the field of treating slight and moderate keratosis lesions. The therapeutic results of the diclofenac treatment are also good, although a complete healing was not yet to be seen because of the high initial number of lesions. It remains to be noted that three patients from the ibuprofen group were completely freed from the optically visible lesions at the end of the 90 days of treatment. The tolerability in both treatment groups was very good.

Other embodiments are within the scope of the following claims.

1. A method for the prevention and treatment of actinic keratosis, comprising administering an effective amount of ibuprofen to a patient in need thereof.

2. The method according to claim 1, wherein administering comprises applying a topical formulation comprising ibuprofen.

3. The method according to claim 2, wherein the topical formulation is in the form of an ointment, a cream, or a gel.

4. The method according to claim 2, wherein the concentration of ibuprofen is from 1 to 20% by weight.

5. The method according to claim 4, wherein the concentration is from 5 to 10% by weight.

6. The method according to claim 5, wherein the concentration is from 6 to 8% by weight.

7. The method according to claim 2, wherein the topical formulation further comprises a penetration enhancer selected from the group consisting of dimethyl isosorbide, poloxamers, laurocapram, pyrrolidones, dimethylsulfoxide, oleic acid, ethylene glycols, derivatives thereof, and mixtures thereof.

8. The method according to claim 2, wherein ibuprofen is the only active ingredient in the topical formulation.

9. The method according to claim 2, wherein the topical formulation further comprises diclofenac.

10. The method according to claim 2, wherein the topical formulation does not include any hyaluronic acid.

11. A pharmaceutical preparation comprising a topical formulation comprising from 6 to 8% by weight of ibuprofen and from 2 to 15% by weight of one or more penetration enhancers.

12. The pharmaceutical preparation according to claim 11, wherein the one or more penetration enhancers includes dimethyl isosorbide.

13. The pharmaceutical preparation according to claim 11, wherein the topical formulation is in the form of a gel.
